(19) United States
(12) Patent Application Publication
(10) Pub. No.: US 2016/0145533 A1
(43) Pub. Date: May 26, 2016

(54) METHOD FOR EXTRACTING ORGANIC SOLIDS AND OIL FROM MARINE ORGANISMS ENRICHED WITH ASTAXANTHIN

(71) Applicants: INSTITUT DE RECHERCHE SUR LES ZONES COTIERES (IRZC), Shippagan (CA); L'ASSOCIATION COOPERATIVE DES PECHEURS DE L'ILSE LTEE, Lamèque (CA)

(72) Inventors: Nadia TCHOUKANOVA, Shippagan (CA); Gerard BENOIT, Lamèque (CA)

(21) Appl. No.: 14/776,481

(22) PCT Filed: Mar. 13, 2014
(86) PCT No.: PCT/CA2014/000251

§ 371 Date: Sep. 14, 2015

Related U.S. Application Data
(60) Provisional application No. 61/782,013, filed on Mar. 14, 2013.

(51) Int. Cl. C11B 3/16; A61K 31/07; A61K 38/17; A61K 31/122; A61K 35/612; C11B 1/10; A61K 31/355

(52) U.S. Cl. CPC C11B 3/16; C11B 1/10; A61K 31/07; A61K 31/355; A61K 31/122; A61K 35/612; A61K 38/1767; A23L 1/3006; A23K 1/164; A23K 1/188; A23V 2002/00

(57) ABSTRACT

A method is for extracting shrimp oil. Particularly, shrimp processing water (EPC) is recovered and subjected to a dissolved air flotation (DAF) system after adding a flocculating agent. The suspended and dissolved solids form aggregates that are recovered from the surface by a procedure referred to as “skimming”. The skimming product is then directed into a horizontal centrifuge (decanter) in order to separate the solid phase (SOC) and the liquid phase having water and shrimp oil. The liquid phase is pumped into the 3-phase vertical centrifuge in order to separate the shrimp oil, the water, and the solids. The solids recovered after using the separator can then be added to the solid phase obtained after settling. The resulting shrimp oil is very rich in astaxanthin and the resulting water contains very little organic material and can be returned to the general processing plant effluent.

### REFERENCES TO THE PREVIOUS APPLICATIONS

The present application claims priority of provisional application U.S. 61/782,013 filed on Mar. 14, 2013, the content of which is entirely incorporated by way of reference.

### FIELD OF THE INVENTION

The present invention relates to a process for extracting organic solids and oil from marine organisms, particularly crustaceans, rich in astaxanthin, and to the astaxanthin-enriched compositions resulting therefrom.

### PRIOR ART

Fishermen's cooperatives and other fishery product processing companies process millions of pounds of shrimp per year. The main final product, the cooked and frozen shrimp, is then distributed to national and international markets. The shrimp processing process comprises several steps (cooking, cooling, shelling, inspection, pickling in brine, draining, freezing, etc.) and requires a very large amount of drinking water (approximately 2000 l/min). Shrimp processing generates an effluent which contains approximately 18 000 mg/l of total solids (TS), consisting of approximately 2000 mg/l of total suspended solids (TSS) and approximately 16 000 mg/l of total dissolved solids (TDS). These particles of raw material contain on average 3000 mg/l of crude proteins and approximately 800 mg/l of fats (oil).

Until very recently, these effluents, considered to be readily biodegradable, were discharged into the environment without any treatment, contributing to a considerable loss of raw material and to pollution of the surrounding coastal waters. In order to remedy this problem, an effluent treatment process and unit have been set up, including, inter alia, a dissolved air flotation (DAF) system. The DAF treatment has made it possible to recover more than 80% of the suspended solids contained in the shrimp effluents in the form of organic sludge. The latter contains at most 8% of solids and 92% of water. This sludge has subsequently been partially dehydrated using a two-phase horizontal centrifuge referred to as a “decanter”. The sludge recovered after using the decanter contains approximately 18% of total solids which consist of less than 2% of fats and of 11% of crude proteins. It is therefore possible to use them as ingredients for feeding animals. Due to the fact that it is the final step of the shrimp effluent treatment, the effluent obtained after the decanter step can now be discharged into the environment.

A sample of this effluent from the decanter taken for analyses particularly attracted our attention because of its vivid pink-orange coloration. Thanks to our expertise in the field of the physicochemical characterization of marine products and derivatives, we understood that this color was due to astaxanthin, a carotenoid pigment which is responsible for the pink-orange coloration of the flesh of salmonids and crustaceans (crab, shrimp, lobster). Surprisingly, we realized that our process also made it possible to concentrate the carotenoid pigments that were dissolved in the water used during the shrimp processing process. In the knowledge that astaxanthin is of great economic interest, the effluent obtained after the decanter step became a subject of detailed studies on our part.

The physicochemical analyses of the decanter effluent demonstrated that it contained a considerable amount of shrimp oil and also of crude proteins. Given that astaxanthin is a pigment that is highly soluble in oil, our objective was to extract the oil from its aqueous medium in order to obtain a product with high added value, rich in astaxanthin. Generally, this pigment can be extracted from the shells of crustaceans by various techniques (e.g. enzymatic digestion) during the production of chitin/chitosan, but the industrial processes used for this operation are normally slow and expensive.

Consequently, the effluent resulting from shrimp processing represents an alternative and very advantageous source for obtaining concentrated astaxanthin in the form of shrimp oil. We have therefore developed a process for extracting astaxanthin-rich shrimp oil from shrimp processing effluent, which, in the past, was discharged into the environment.

### SUMMARY OF THE INVENTION

In accordance with a first aspect, the invention relates to a process for extracting oil and organic solids from a marine organism, particularly an oil and an organic solid which are enriched with active ingredient, inter alia astaxanthin.

In accordance with one particular aspect, the invention relates to a process for extracting oil of marine origin, comprising:
a) obtaining a marine organism treatment effluent;
b) adding a flocculating agent to the treatment liquid of a) and separating an aqueous phase from the solids flocculated at the surface in order to recover the solids therefrom;
c) separating the solids recovered in b) into a solid phase and a liquid phase and recovering the solid phase and/or the aqueous phase;
d) subjecting the liquid phase obtained in c) to a vertical centrifugation in order to obtain an aqueous phase and an oil; and
e) recovering the oil thus separated.

Particularly, in step b), the separation of the aqueous phase from the solids flocculated at the surface is carried out with a dissolved air flotation (DAF) system; in step c), the separation of said solids flocculated in b) into a solid phase and a liquid phase is carried out using a two-phase horizontal centrifuge (decanter); and in step d), the obtaining of the aqueous phase and of the oil is carried out by subjecting the liquid phase obtained in c) to a vertical centrifugation (separator).

In accordance with another aspect, the process also comprises the following step:
f) the solid phase recovered in step c) is then dried in order to form a protein-enriched organic solid.

In accordance with one particular aspect, the invention relates to the oil as obtained by means of the process presently defined. Particularly, the shrimp oil comprises more than 800 μg/g (ppm) of astaxanthin, more than 500 μg/g of vitamin E, more than 2000 IU/100 g of vitamin A, and more than 13 g/100 g of ω-3 fatty acids. In accordance with one particular aspect, the invention also relates to a solid residue as obtained by means of the process presently defined. Particularly, the solid residue comprises more than 60% proteins, more than 400 μg/g of vitamin E, more than 4000 IU/100 g of vitamin A, and more than 350 μg/g of astaxanthin. Alternatively, the invention relates to a composition comprising an oil and/or an organic solid as presently defined, mixed with an excipient. Particularly, the excipient is a meal, particularly a meal enriched with marine proteins.

In accordance with one particular aspect, the invention also relates to the use of an oil or of the solid residue as presently defined, as a food additive in an agriculture feed.

Likewise, the invention relates to the use of an oil or of the organic solids as presently defined, for the production of a food or of a food supplement. Particularly, the food or the food supplement is intended for human, animal (such as farm animals, domestic animals) or aquaculture use. More particularly, the food additive is used in an aquaculture feed.

Further, in accordance with one particular aspect, the invention also relates to the use of an oil or of the solid product as presently defined, as a food additive in foods intended for birds such as laying hens and other poultry.

### DETAILED DESCRIPTION OF THE INVENTION

### Description of the Figures

FIG. 1. Diagram of the shrimp oil extraction process.

FIG. 2. Variation in the concentration of crude proteins in the sludge recovered from the decanter as a function of the increase in the rotational speed of the decanter bowl during experiments A, B and C.

FIG. 3. Variation in the concentration of the fats in the sludge recovered from the decanter as a function of the increase in the rotational speed of the decanter bowl during experiments A, B and C.

FIG. 4. Variation in the concentration of the total solids in the effluent of the decanter as a function of the increase in the rotational speed of the decanter bowl during experiments A, B and C.

FIG. 5. Variation in the concentration of the total suspended solids in the effluent of the decanter as a function of the increase in the rotational speed of the decanter bowl during experiments A, B and C.

FIG. 6. Variation in the concentration of the crude proteins in the effluent of the decanter as a function of the increase in the rotational speed of the decanter bowl during experiments A, B and C.

FIG. 7. Variation in the concentration of the fats in the effluent of the decanter as a function of the increase in the rotational speed of the decanter bowl during experiments A, B and C.

FIG. 8: Effect of the temperature and of the speed of the decanter on the concentration of fat (F) in the effluent of the decanter.

FIG. 9: Effect of the temperature and of the speed of the decanter on the concentration of total solids (TS) in the effluent of the decanter.

FIG. 10: Effect of the temperature and of the speed of the decanter on the percentage of total solids (TS) and of fats (F) in the shrimp organic solids (SOC).

FIG. 11: Effect of the SKIM temperature on the concentration of total solids and of fats of decanter effluent when the decanter rotates at 2700 rpm.

FIG. 12: Effect of the temperature on the percentage of total solids and of fats in the SOC.

### ABBREVIATIONS AND DEFINITIONS

### Abbreviations

DAF: dissolved air flotation; DEC: 2-phase horizontal decanter or centrifuge; EC: heat exchanger; EPC: shrimp processing process water: cooking water, rinsing water, etc.; PL-E: liquid phase; PS-RC: solid phase; HC: shrimp oil; MO: organic material; SEP: vertical separator or centrifuge; R1: receptacle 1; R2: receptacle 2; TDS: total dissolved solids; SKIM: layer of flocculated solids skimmed at the surface of the liquid phase; SOC: shrimp organic solids; TSS: total suspended solids; and TS: total solids.

### Definitions

The use of the expression “approximately” as used in the present document refers to a margin of error of + or −5% of the number indicated. To be more precise, the term “approximately” when used, for example, with the term 90%, means 90%+/−4.5%, i.e. from 86.5% to 94.5%.

The term “solid residue” as used in the present document refers to a “protein concentrate” of shrimp or another marine organism resulting from the present process, and these two terms can be used interchangeably.

### Detailed Description of Particular Implementations

The present invention relates to the exploitation of the waste water effluents from the processing and production of marine organisms such as fish (inter alia fatty fish) and crustaceans (including, inter alia, herring, sardine, mackerel, salmon, trout, shrimp, crab, lobster and krill). The food processing of these organisms is responsible for unsuspected pollution of coastal areas and the applicant has entirely fortuitously found that a method for reducing the organic load of processing plant effluents makes it possible to isolate an oil and a meal from these marine organisms, which are greatly enriched with active ingredients and thus have a strong added value.

### Shrimp Processing

The shrimp processing process comprises several steps (cooking, cooling, shelling, inspection, pickling in brine, draining, freezing, etc.) and requires a very large amount of drinking water (approximately 2000 l/min). Shrimp processing generates an effluent which contains approximately 18 000 mg/l of total solids (TS), consisting of approximately 2000 mg/l of total suspended solids (TSS) and approximately 16 000 mg/l of total dissolved solids (TDS). These particles of raw material contain on average 3000 mg/l of crude proteins and approximately 800 mg/l of fats (oil).

Until very recently, these effluents, considered to be readily biodegradable, were discharged into the environment without any treatment, contributing to a considerable loss of raw material and to pollution of the surrounding coastal waters. In order to remedy this problem, the applicant has used an effluent treatment process and unit, including, inter alia, a dissolved air flotation (DAF) system. The DAF treatment has made it possible to recover more than 80% of the suspended solids contained in the shrimp effluents in the form of organic sludge. The latter contains at most 8% of solids and 92% of water. This sludge has subsequently been partially dehydrated using a two-phase horizontal centrifuge referred to as a “decanter”. The sludge recovered after using the decanter contains approximately 18% of total solids which consist of less than 2% of fats and of 11% of crude proteins. It is therefore possible to use them as ingredients for feeding animals. Due to the fact that it is the final step of the shrimp effluent treatment, the effluent obtained after the decanter step can now be discharged into the environment.

During an unforeseen event (break), a sample of this effluent from the decanter taken for analyses particularly attracted our attention because of its vivid pink-orange coloration. Following a physicochemical characterization of the marine products and derivatives, we established that this color was due to astaxanthin, a carotenoid pigment that is responsible for the pink-orange coloration of the flesh of salmonids and crustaceans (crab, shrimp, lobster). Surprisingly, we realized that our process also made it possible to concentrate the carotenoid pigments that were dissolved in the water used during the shrimp processing process. In the knowledge that astaxanthin is of great economic interest, the effluent obtained after the decanter step became a subject of detailed studies on our part.

The physicochemical analyses of the decanter effluent demonstrated that it contained a considerable amount of shrimp oil and also of crude proteins. In the knowledge that astaxanthin is a pigment that is highly soluble in oil, our objective was to extract the oil from its aqueous medium in order to obtain a product with a high added value, rich in astaxanthin. Generally, this pigment can be extracted from the shells of crustaceans during the production of chitin/chitosan by enzymatic digestion, but this industrial process is very slow and very expensive.

### The Process

In accordance with one particular aspect, the invention relates to a process for extracting oil from a crustacean, comprising:
a) obtaining a marine organism treatment effluent;
b) adding a flocculating agent to the treatment liquid of a) and separating an aqueous phase from the solids flocculated at the surface in order to recover the solids therefrom;
c) separating said solids recovered in b) into a solid phase and a liquid phase and recovering the solid phase and/or the aqueous phase;
d) subjecting the liquid phase obtained in c) to a vertical centrifugation in order to obtain an aqueous phase and an oil; and
e) recovering the oil thus separated.

Particularly, in step b), the separation of the aqueous phase from the solids flocculated at the surface is carried out with a dissolved air flotation (DAF) system; in step c), the separation of said solids flocculated in b) into a solid phase and a liquid phase is carried out using a 2-phase horizontal centrifuge referred to as a decanter (DEC); and in step d), the obtaining of the aqueous phase and of the oil is carried out by subjecting the liquid phase obtained in c) to a vertical centrifugation known as a separator (SEP).

### Flocculating Agent

Flocculation consists of a process of agglomeration of solid particles (fats and crude proteins) around the flocculating agent which has an opposite charge. The products used are preferably recognized as safe (Generally Recognized as Safe; GRAS). According to one particular implementation, the flocculation agent added to the shrimp treatment liquid is chosen from: natural or synthetic polymers, whether they are cationic (positively charged) or anionic (negatively charged).

The recovery of the organic material (protein and oil) from the marine organism processing effluent with anionic flocculating agent comprises three steps: acidification (addition of a sulfuric acid), coagulation (addition of a coagulant; FeCl₃ or AlCl₃) and flocculation (addition of an anionic flocculating agent such as polyacrylamide or alginate), while cationic flocculating agents do not require any pretreatment of the effluent. Particularly, the anionic flocculating agent is chosen from: Polyfloc AP1110 (from Ge Water Technologies). More particularly, the flocculating agent chosen is a cationic flocculating agent, such as polyacrylamide or chitosan. Even more particularly, the cationic flocculating agent is chosen from: Polyfloc CP1158 (from Ge Water Technologies) and GR-505 (from Nalco).

### DAF

According to one particular implementation of the invention, the separation of the flocculated solids from the solvent in step b) is carried out by virtue of a dissolved air flotation (DAF) system. According to this implementation, the dissolved air attaches to the flocculated solids (“flocs”) and brings them to the surface where they float and are then recovered by means of a procedure referred to as skimming, according to which the upper layer of the aggregates (flocculated solids) at the surface is scraped.

The treatment liquid is usually at an ambient temperature or else at approximately 10 to 18° C. when it reaches the dissolved air flotation system.

Particularly, the flocculating agent is added during the transfer of the liquid between the reservoir and the entry into the DAF system.

### Decanter (DEC)

According to one particular implementation of the invention, the separation of the flocculated solids recovered in step b) into a solid phase and a liquid phase is carried out by virtue of a 2-phase horizontal centrifuge system, commonly referred to as “decanter” or DEC. Particularly, this decanter has two continuous outlets, including one outlet for the solid phase and one outlet for the liquid phase consisting of water (effluent) and of shrimp oil.

According to this implementation, the liquid phase is recovered following decanting (horizontal centrifugation) with a bowl rotational speed between 1800 rpm and 3300 rpm. More particularly, the decanting is operated at a speed between 2000 and 2900 rpm, even more particularly between 2500 and 2800 rpm, and even more particularly around 2700 rpm.

### Centrifuge (SEP)

According to one particular implementation of the invention, the separation of the effluent and the oil from the liquid phase obtained at the decanter outlet is carried out by virtue of a vertical centrifuge (commonly referred to as “separator”) where the oil is recovered above the effluent (aqueous phase). Particularly, this separator operates continuously and comprises 3 outlets: the top outlet (supernatant); the center outlet (centrate) and the bottom outlet (pellet). Particularly, the supernatant contains the oil; the “centrate” consists of water virtually free of organic materials and can now be discharged into the processing plant effluent without any danger to the environment; and the pellet contains a solid phase which may be recovered in order to enrich a meal with proteins.

### Heat Exchangers

According to one particular implementation of the invention, each separation step is carried out at a pre-established temperature in order to optimize the separation of the desired components. Particularly, in step a), the treatment of the shrimps is carried out at high temperature in order to cook the shrimps; more particularly, the cooking water is at a temperature of approximately 100° C. The cooking water is then mixed with a large amount of cold water used for cooling and shelling the shrimp in the receptacle R1 in order to reach a temperature of approximately 4 to 25° C., more particularly between 8 and 20° C., and even more particularly between 10 and 18° C. at the time of the entry into the DAF system (step b).

According to one particular implementation of the invention, this separation step is carried out at a pre-established temperature in order to optimize the separation of the desired components. Particularly, heat exchangers can be installed in order to control the temperature of the solid and/or liquid phase in order to thereby optimize the separation of the components. More particularly, once step b) has been completed and the solid phase has been recovered (skims), the latter is subjected to a first heat exchanger (EC1) in order to control the temperature thereof before carrying out the decanter (DEC) step. More particularly, the skimmed solids (skims) are subjected to a heat exchanger in order to thereby raise their temperature to about 20 to 40° C., more particularly around 25 to 35° C., and even more particularly approximately 30° C.

Particularly, once step c) has been completed and the liquid phase has been recovered (HC+E), the latter is subjected to a second heat exchanger (EC2) in order to control the temperature thereof before carrying out the separator (SEP) step. More particularly, the oil+water mixture is subjected to a second heat exchanger in order to thereby raise their temperature to about 80 to 99° C., more particularly around 85 to 98° C., and even more particularly approximately 95° C. before carrying out the separation in the vertical centrifuge.

Alternatively, if the objective of the present method is instead to recover the organic material in the solid phase rather than in the liquid phase, once step b) has been completed and the solid phase has been recovered (skims), the latter is subjected to a first heat exchanger (EC1) in order to increase the temperature thereof before carrying out the decanter (DEC) step. Thus, more particularly, the skimmed solids (skims) are subjected to a heat exchanger in order to raise the temperature thereof to about 70 to 95° C., more particularly around 80 to 90° C., and even more particularly approximately 85° C. in order to facilitate the migration of the organic material to the solid phase.

### Particular Implementation of the Process

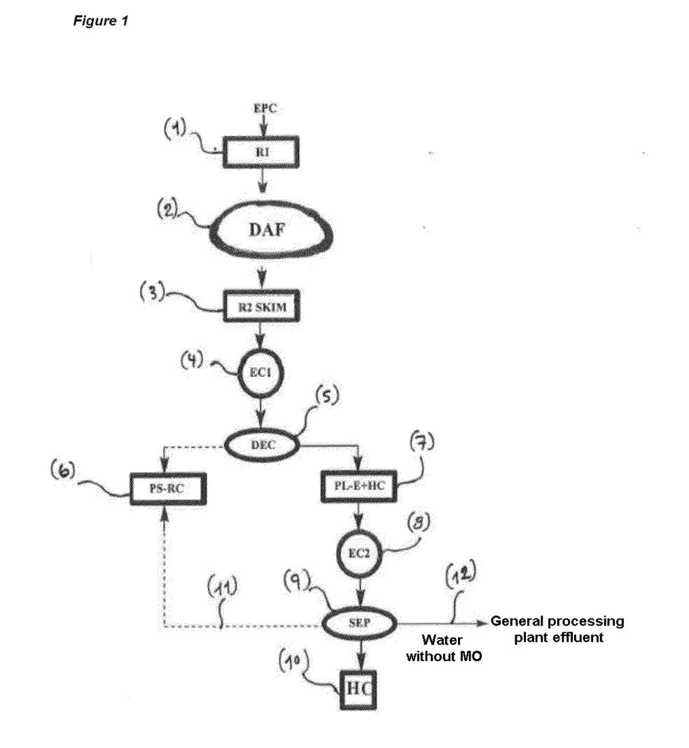

The shrimp oil extraction process comprises several steps and requires several pieces of equipment. The diagram of a particular implementation of the shrimp oil extraction process is presented in FIG. 1.

The water which leaves the shrimp processing process (EPC) is first recovered in a reservoir (R1) (1) for homogenization, and is then pumped at a flow rate of approximately 2000 l/min into a dissolved air flotation (DAF) system (2). The EPC temperature is varied from 10 to 18° C. A cationic flocculating agent (or anionic flocculating agent with a) acidification and b) coagulation beforehand) is added to the EPC so that the suspended and dissolved solids form larger flocs (aggregates). In the DAF system (2), the dissolved air attaches to the flocculated solids and brings them to the surface where they are recovered by means of a procedure referred to as “skimming”. The solids recovered, referred to as “skimmings” (SKIMs), are recovered in a second reservoir (R2 SKIM) (3). In this step, the SKIMs contain approximately 6% of total solids, the oil and 94% of water.

The SKIMs are first pumped at 50 l/min into a heat exchanger (EC1) (4) in order to raise the temperature thereof to approximately 30° C. (80 to 100° F.), and are then directed into the horizontal centrifuge (DEC) (5). The latter is used to separate the solid phase (shrimp organic solids (PS-RC) (6)) and the liquid phase which is recovered in a third reservoir (PL-E+HC) (7). This liquid phase (7) consists of water and of shrimp oil having a few traces of crude proteins. In order to separate the shrimp oil from the water, the liquid phase is pumped first to a second heat exchanger (EC2) (8) in order to raise its temperature to approximately 95° C. and then to the 3-phase vertical centrifuge (SEP) (9) with 3 continuous outlets. This centrifuge (SEP) makes it possible to separate the shrimp oil (upper phase), the water/effluent (middle phase), and the solid residue (lower phase). These recovered solids (11) are then added to the solid phase (6) obtained after the decanting. The shrimp oil obtained (upper phase) is recovered in metal drums under nitrogen (HC) (10), and is stored in a warehouse refrigerated at −18° C. The water obtained (12) containing very little organic material (Water without MO) is returned to the general processing plant effluent.

### Oil/Solid Obtained and Compositions

Unexpectedly, the implementation of this process produces an oil very rich in astaxanthin, that is to say containing more than 505 μg/g of astaxanthin, particularly more than 510, 525, 550, 580, particularly more than 600 μg/g, more than 700 μg/g, more than 800 μg/g, more than 900 μg/g, or else more than 1000 μg/g of astaxanthin. Particularly, this oil comes mainly from shrimp.

Likewise, the present process makes it possible to obtain a solid product comprising more than 60% proteins, more than 12% of fat, more than 400 μg/g of vitamin E, more than 4000 IU/100 g of vitamin A, and more than 350 μg/g of astaxanthin. Particularly, this residue is referred to as “marine protein concentrate”. Particularly, this concentrate comes mainly from shrimp. More particularly, this concentrate is dried and optionally milled in order to make therefrom a meal enriched with marine proteins.

According to one particular implementation, the invention also relates to a composition comprising the oil as presently defined, mixed with an excipient.

Particularly, the excipient may be a solid residue, a concentrate or a meal, more particularly a meal enriched with marine proteins.

According to one particular implementation, the invention also relates to a feed intended for feeding at least one farm-raised fish, said feed comprising an oil or else a solid residue as presently defined.

According to one particular implementation, the invention also relates to a food supplement comprising an oil or a solid residue as presently defined, both or either being mixed with a physiologically acceptable excipient, particularly for human beings.

### Uses

According to one particular implementation, the invention also relates to the use of an oil as presently defined, as a food additive in an aquaculture feed (i.e. for farm-raised fish) or poultry feed, particularly the use of this oil for the production of a food or of a food supplement. Particularly, the food or the food supplement is intended for human, animal, aquaculture or poultry use. Alternatively, the invention relates to the use of a solid product (i.e. residue or concentrate) as presently defined, as a food additive in an aquaculture and/or poultry feed.

Particularly, the food supplement or additive is designed to be ingested in liquid or solid form by human beings or animals, such as farm animals or domestic animals, or else by birds or fish.

### Methods

According to one particular implementation, the invention also relates to a method for feeding a farm-raised fish, said method comprising the administration of the feed as presently defined.

Likewise, in another particular implementation, the invention relates to a method for combating free-radical oxidation in human beings, said method comprising the administration of a dose effective against oxidation of the food supplement as presently defined.

The following examples are only by way of illustration, rather than limiting the invention to these particular implementations.

### EXAMPLES

### Example 1

### Material

The dissolved air flotation system is of the Krofta brand, model Multifloat, MFV-600 Tandem; the decanter (DEC) is of the Sharples brand, model Super D-Canter, P3400; and the centrifuge (SEP) is obtained from Alpha Laval, model AFPX-409.

### Example 2

### Determination of the Optimal Conditions for Operating the Decanter Making it Possible to Extract the Maximum Amount of Oil and the Minimum Amount of Proteins in the Shrimp Effluent

The objective of the first step of this project is to find the optimal conditions for the two-phase horizontal centrifuge (decanter) in order to obtain a greater amount of oil and a smaller amount of proteins in the decanter effluent without harming the quality of the organic sludge recovered. The rotational speed of the decanter bowl is a parameter which affects the separation of the solids from the liquid. For this, a “gearbox” was installed on the decanter in order to control the rotational speed of the bowl. Three experiments were carried out on three different shrimp processing days in order to obtain more representative results and to draw reliable conclusions. During the first two experiments (A and B), the decanter operation efficiency was evaluated at 7 different rotational speeds of the decanter bowl, defined in consultation with the ACPI technical team. The third experiment C was carried out in order to confirm the results obtained during the first two experiments. The course of these experiments is described in Table 1.

TABLE 1
Course of the experiment for evaluating the
decanter conditions during experiments A and B

| | Speed (RPM) | | | | | | |
|---|---|---|---|---|---|---|---|
| | 1800 | 2160 | 2520 | 2700 | 2880 | 3060 | 3240 |
| | Time | | | | | | |
| | 8 h 30 | 9 h 30 | 10 h 30 | 11 h 30 | 12 h 30 | 13 h 30 | 14 h 30 |
| 1^{st} subsamples | 9 h 00 | 10 h 00 | 11 h 00 | 12 h 00 | 13 h 00 | 14 h 00 | 15 h 00 |
| 2^{nd} subsamples | 9 h 15 | 10 h 15 | 11 h 15 | 12 h 15 | 13 h 15 | 14 h 15 | 15 h 15 |
| 3^{rd} subsamples | 9 h 30 | 10 h 30 | 11 h 30 | 12 h 30 | 13 h 30 | 14 h 30 | 15 h 30 |
| Composite | DAF INF | Dec EFF | Dec EFF | Dec EFF | Dec EFF | Dec EFF | DAF INF |
| samples | DAF EFF | Dec SLUDGE | Dec SLUDGE | Dec SLUDGE | Dec SLUDGE | Dec SLUDGE | DAF EFF |
| | Dec EFF | | | | | | Dec EFF |
| | Dec SLUDGE | | | | | | Dec SLUDGE |

At each speed, the decanter was operated for half an hour before taking the first subsamples. Fifteen minutes later, the second subsamples are taken. Then, after a further 15 minutes of operation, the third subsamples are taken. For each rotational speed of the decanter bowl, subsamples were taken at the following places: 1) the DAF influent and effluent (DAF INF, DAF EFF), 2) the decanter effluent (Dec EFF) and 3) the decanter sludge (Dec SLUDGE). A composite sample was prepared from 3 subsamples of each matrix sampled for each rotational speed of the decanter and the physicochemical analyses were carried out.

In order to be sure that the quality of the shrimp effluent and the DAF operating efficiency are stable, the composite samples of DAF influent and effluent of the three experiments were analyzed and the results of these analyses are given in Table 2.

TABLE 2
Results of the evaluation of the physicochemical parameters
of the shrimp processing effluent (DAF INF), of the DAF
effluent (DAF EFF) and of the DAF operating efficiency
during the experiments of Jun. 9, 19 and 28, 2006

| | Analyses | | | |
|---|---|---|---|---|
| | Total sus- | Total | Crude | |
| | pended solids | nitrogen | proteins | Fats |
| Sample | (TSS) mg/l | (TKN) mg/l | (CP) mg/l | (F) mg/l |
| DAF INF exp. A | 2191 | 462 | 2890 | 1099 |
| DAF EFF exp. A | 476 | 333 | 2079 | 443 |
| % Reduction | 78% | 28% | 28% | 60% |
| DAF INF exp. B | 1404 | 434 | 2713 | 1013 |
| DAF EFF exp. B | 276 | 293 | 1831 | 312 |
| % Reduction | 80% | 32% | 32% | 69% |
| DAF INF exp. C | 2007 | 400 | 2500 | 1052 |
| DAF EFF exp. C | 294 | 289 | 1806 | 341 |
| % Reduction | 85% | 28% | 28% | 68% |

The results of the analyses of the DAF influent and effluent samples show that the average content of total suspended solids (TSS) varied slightly, while those of the total nitrogen (TKN), of the crude proteins (CP) and of the fats (F) were similar during the three experiments. The operation of the DAF was more efficient during experiments B and C, especially with regard to the recovery of the TSS (80% and 85% were recovered, respectively) and of the fats (69% and 68% were recovered, respectively). The percentages of reduction of the TSS (78% to 85%), TKN (28% to 32%), CP (28% to 32%) and F (60% to 69%) in the shrimp processing effluent demonstrate that the DAF system operates with the same efficiency that was obtained in the previous year after its optimization was finalized.

The composite samples of the organic sludge recovered from the decanter during the three experiments were analyzed in order to observe the change in the quality of the organic solids as a function of the various speeds (RPM) of the decanter bowl. The results of these analyses are given in Table 3.

TABLE 3
Results of evaluation of the physicochemical parameters of the
decanter sludge at various rotational speeds of the decanter
bowl during the experiments of Jun. 9, 19 and 28, 2006

| Decanter | Moisture | | | Total solids | | | Crude proteins | | | Fats | | |
|---|---|---|---|---|---|---|---|---|---|---|---|---|
| speed | content % | | | (TS) % | | | (CP) % | | | (F) % | | |
| (RPM) | A | B | C | A | B | C | A | B | C | A | B | C |
| 1800 | 83.34 | 82.11 | | 16.66 | 17.89 | | 9.94 | 10.19 | | 3.08 | 3.68 | |
| 2160 | 83.19 | 82.80 | | 16.81 | 17.20 | | 10.44 | 10.69 | | 2.73 | 2.84 | |
| 2520 | 82.93 | 82.62 | 82.98 | 17.07 | 17.38 | 17.02 | 10.81 | 10.38 | 10.81 | 2.49 | 2.11 | 2.19 |
| 2700 | 82.57 | 82.50 | 82.88 | 17.43 | 17.50 | 17.12 | 10.94 | 11.00 | 10.88 | 2.22 | 2.00 | 2.29 |
| 2880 | 82.70 | 82.56 | 82.50 | 17.30 | 17.44 | 17.50 | 11.25 | 10.88 | 11.13 | 1.95 | 2.21 | 1.66 |
| 3060 | 80.91 | 82.27 | | 19.09 | 17.73 | | 11.38 | 11.25 | | 1.76 | 2.52 | |
| 3240 | 81.43 | 82.22 | | 18.57 | 17.78 | | 11.63 | 11.94 | | 1.83 | 2.22 | |

The results of the analyses demonstrated that, with the increase in the rotational speed of the decanter bowl, the percentage of total solids in the sludge increased, whereas the percentage moisture content slightly decreased in experiment A, but the contents of these two parameters remained stable in experiments B and C.

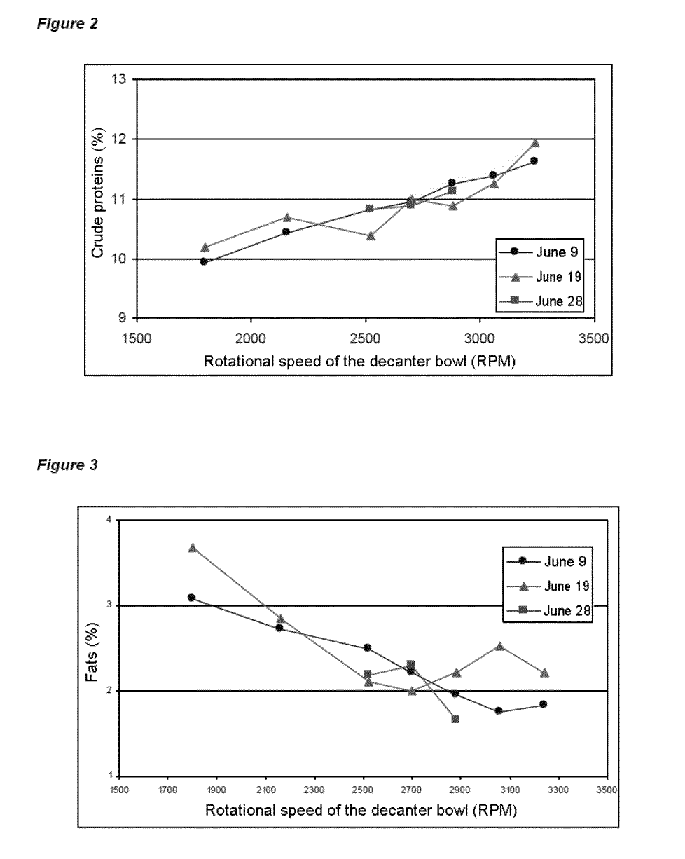

FIG. 2 clearly demonstrates that, with the increase in the rotational speed of the decanter, a slight increase in the content of crude proteins is noted in the sludge recovered, thus causing an improvement in the nutritional value of the sludge. It should be emphasized that the proteins are the main constituent desired if the sludge is used as an additive for feeding animals or fish.

The results of the analyses showed that, with the increase in the rotational speed of the decanter bowl, the percentage of fats (F) in the sludge recovered decreases (Table 3). For example, during the experiment of June 9, the F content decreased from 3.08% to 1.83% when the rotational speed of the decanter bowl was increased from 1800 rpm to 3240 rpm. The results of the analyses of June 19 confirmed this trend. FIG. 3 clearly shows that, during experiments A and B, a rapid decrease in F is observed with the increase in the rotational speed of the decanter from 1800 to 2700 rpm.

When the rotational speed of the decanter exceeds 2700 rpm (75% of the maximum speed), the results obtained are non-conclusive. The F content continues to decrease for experiment A, whereas for experiment B, an increase in the F contents is observed, followed by a decrease when the rotational speed of the decanter reaches 3240 rpm.

It should be specified that this slight decrease in F in the sludge recovered does not affect its quality since a very high F concentration can be harmful during the drying of the sludge. On the other hand, the F which are so to speak lost in the sludge are found in the decanter effluent, thereby increasing our chance of recovering them in the form of oil using the separator. Thus, the results of the analyses demonstrated that it is possible to change the rotational speed of the decanter bowl without significantly harming the quality of the organic sludge recovered.

In order to target more exactly the optimal speed of the decanter that makes it possible to obtain a greater amount of oil and a smaller amount of proteins in the decanter effluent, the composite samples of the decanter effluent of the three experiments A, B and C were analyzed. The contents of total solids, total suspended solids, crude proteins and fats were determined and the results of these analyses are given in Table 4.

TABLE 4
Results of the evaluation of the physicochemical parameters of the
composite samples of the decanter effluent at various rotational
speeds of the decanter bowl during experiments A, B and C

| | Total suspended | | | | | | | | | | | |
|---|---|---|---|---|---|---|---|---|---|---|---|---|
| Decanter | solids (TSS) | | | Total solids | | | Crude proteins | | | Fats (F) | | |
| speed | mg/l | | | (TS) mg/l | | | (CP) mg/l | | | mg/l | | |
| (RPM) | A | B | C | A | B | C | A | B | C | A | B | C |
| 1800 | 18 533 | 21 850 | | 31 380 | 38 025 | | 3731 | 3381 | | 16 030 | 21 390 | |
| 2160 | 25 025 | 23 500 | | 38 560 | 41 058 | | 4519 | 4150 | | 21 610 | 23 115 | |
| 2520 | 30 250 | 30 300 | 23 050 | 42 455 | 45 469 | 40 475 | 7319 | 5169 | 4350 | 22 760 | 26 720 | 22 840 |
| 2700 | 33 500 | 30 700 | 29 550 | 45 226 | 45 456 | 44 649 | 6956 | 5125 | 4794 | 25 110 | 26 440 | 24 300 |
| 2880 | 37 900 | 36 500 | | 49 152 | 50 393 | | 11 550 | 7069 | | 24 220 | 29 870 | |
| 3060 | 33 500 | 37 200 | 30 200 | 45 188 | 52 464 | 45 323 | 7975 | 7838 | 6994 | 23 520 | 28 260 | 23 640 |
| 3240 | 34 200 | 41 300 | | 45 655 | 55 123 | | 11 275 | 8881 | | 20 860 | 28 750 | |

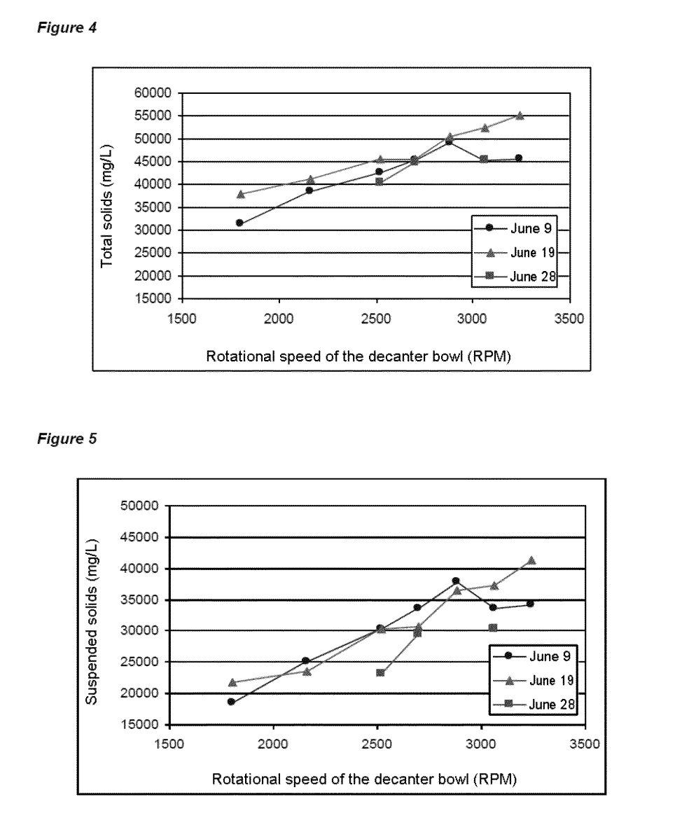

When examining the results of the analyses carried out on the composite samples of the decanter effluent, it is noted that the concentrations of the solids (TS and TSS), of the crude proteins (CP) and of the fats (F) in the effluent increase with the increase in the rotational speed of the decanter bowl. The increase in the contents of the solids (TS and TSS), of the crude proteins (CP) and of the fats in the effluent with the increase in the rotational speed of the decanter bowl results in a decrease in the amount of organic sludge recovered without, however, harming its quality. FIGS. 4 and 5 confirm this migration of the solids into the decanter effluent.

During the tests of experiment A, the concentration of the solids (TS and TSS) in the effluent increased in a constant and rapid manner with the increase in the rotational speed of the decanter bowl. The concentration of the solids reached its maximum (37 900 and 49 152 mg/l of TSS and TS, respectively) at a decanter bowl speed of 2880 rpm. Subsequently, the TSS and TS contents in the effluent decreased when the decanter bowl speed was further increased. On the other hand, during experiment B, a constant increase in the solids (TS and TSS) in the effluent was noted with the increase in the rotational speed of the decanter bowl. For example, the effluent sampled at the highest rotational speed of the decanter, i.e. 3240 rpm, contained TSS and TS contents of 41 300 and 55 123 mg/l, respectively. It should be emphasized that a high concentration of solids in the effluent can be harmful to the correct operating of the separator and can prevent the extraction of the oil from the aqueous medium in addition to reducing the sludge recovery yield. The decanter effluent samples taken at bowl rotational speeds greater than 2700 rpm (75% of maximum operating speed) are less suitable for optimal extraction of the oil from the decanter effluent since they contain too great an amount of solids.

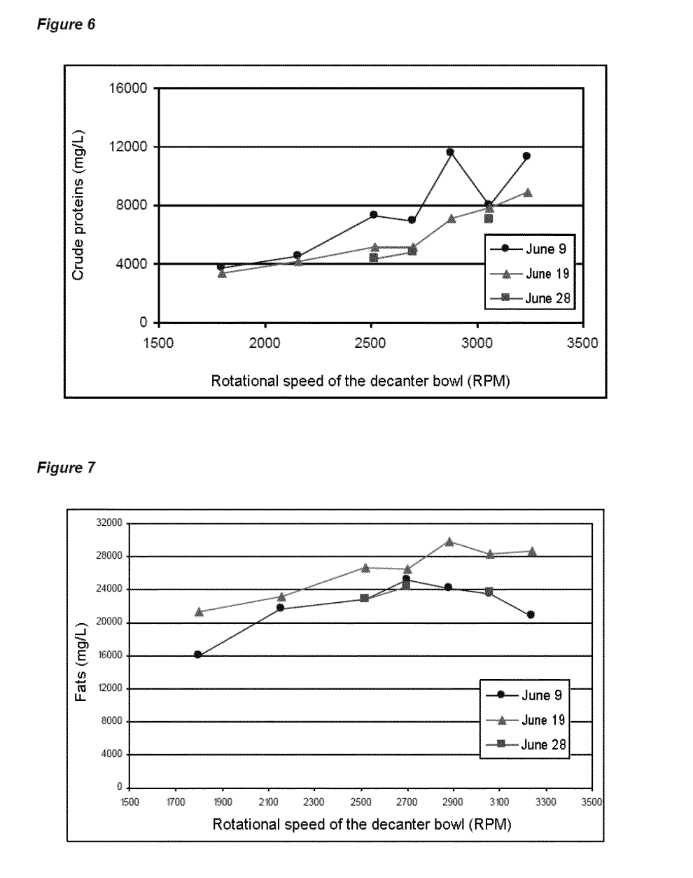

FIG. 6 shows that the variation in the concentration of crude proteins in the decanter effluent as a function of the increase in the rotational speed of the decanter bowl follows the same trend observed for the solids (TS and TSS).

According to FIG. 6, it is observed that the content of crude proteins (CP) in the decanter effluent increases with the increase in the rotational speed of the decanter bowl, this being for all the tests (A, B and C). The migration of the CP into the effluent definitely becomes significant when the speed of the decanter bowl reaches 2700 rpm, i.e. 75% of the maximum operating speed of the decanter. Due to the fact that the proteins are the main factor that can negatively influence the extraction of the shrimp oil from the decanter effluent, it is preferable to work with rotational speeds of less than 2700 rpm.

The results given in Table 4 demonstrate that all the samples taken during experiments A, B and C were very rich in fats. The lowest F content was obtained during the tests A using a decanter rotational speed of 1800 rpm and was 16 030 mg/l. When the rotational speed of the decanter bowl is increased to 2160 rpm (60% of the maximum operating speed), the F content in the effluent reaches a maximum of 25 110 mg/l. It should also be noted that the F content greatly exceeds that of the crude proteins in all the samples analyzed. FIG. 7 shows that the variation in the concentration of the fats in the decanter effluent as a function of the increase in the rotational speed of the decanter bowl follows the same trend observed for the solids (TS and TSS) since it is their main constituent.

The F concentration in the effluent increased with the increase in the speed and reached a maximum of 25 110 mg/l at a rotational speed of the decanter bowl of 2700 rpm (A) and 29 870 mg/l at a rotational speed of the decanter bowl of 2880 rpm (B). When the rotational speed of the decanter bowl is further increased, the fat content in the effluent begins to decrease.

The results of these experiments demonstrated that:
The quality of the shrimp effluent varied only slightly during these experiments.
The change in the rotational speed of the decanter bowl did not significantly affect the quality of the organic sludge recovered.
The increase in the rotational speed of the decanter bowl promotes an increase in the solids (TS and TSS), the crude proteins and the fats in the effluent.
An excessive decanter rotational speed can have a negative impact on the extraction of the oil and on the yield of the organic sludge recovered.
The decanter effluent samples that were taken during the operation of the decanter at bowl rotational speeds greater than 2700 rpm are not suitable for the production of shrimp oil since they contain too great an amount of solids and of proteins.
The samples that were treated by operating the decanter at a rotational speed of 1800 rpm (the slowest speed tested in this project) have a lower concentration of fats despite the fact that their solids and protein contents are perfect for operating the separator.
The decanter effluent samples that were taken during the operation of the decanter at bowl rotational speeds of 2160, 2520 and 2700 rpm (i.e. 60%, 70% and 75% of the maximum speed, respectively) appear to be the most suitable for the extraction of the shrimp oil since they contain a good amount of fats and moderate amounts of solids and especially of proteins.

### Example 3

### Optimization of the Conditions for Extraction of the Astaxanthin-Rich Shrimp Oil from the Decanter Effluent Using the Separator

It is important to recall that the decanter effluent constitutes the final effluent of the process for treating the effluent resulting from the shrimp processing before discharge of said effluent into the environment. It is also at the level of the decanter that the organic material that is contained in the effluent is recovered. Consequently, our objective is therefore to develop a process which makes it possible to extract the oil and the residual proteins that are contained in the decanter effluent.

The extraction of herring oil from its aqueous medium by centrifugation is a process which is already used in the production of herring meal and oil. A used separator was adapted in order to carry out our tests for extraction of shrimp oil from the decanter effluent. The conditions which have the greatest effect on the separation of the oil from its aqueous medium are:
the effluent centrifugation speed;
the amount of solids (especially suspended solids) in the effluent;
the flow rate of the effluent to be centrifuged; and
the effluent temperature.

Given that the operating speed of the separator cannot be varied, all efforts are concentrated on the optimization of the other three parameters, such as the amount of solids in the effluent, its flow rate and its temperature at the separator inlet. According to the results of the first step, it was demonstrated that the decanter effluent samples that were taken at bowl rotational speeds of 2160, 2520 and 2700 rpm appear to be the most suitable for recovering the oil that they contain since they contain a considerable amount of fats while at the same time having moderate amounts of solids and of proteins. After an exhaustive analysis of all the data that were available for the preliminary tests, it was decided to extract the shrimp oil from the effluents that come from the decanter at a bowl rotational speed of 2700 rpm. The first test (D) was carried out under the following conditions:
1) the rotational speed of the decanter bowl was 2700 rpm;
2) the decanter effluent was pumped into the separator with a flow rate of 38 liters per minute (2280 liters per hour). This flow rate corresponds to 45% of the maximum speed of the pump; and
3) the effluent was used at ambient temperature.

As early as this first test D, it was possible to successfully extract shrimp oil. By centrifuging 1634 liters of effluent, 18.5 kg of oil was extracted, which represents an average recovery rate of 11.3 grams of oil per liter of effluent. In the knowledge that the decanter effluent can contain between 24 and 26 g of oil per liter of effluent (see Table 4), the extraction yield was consequently considered to be insufficient. Once the 18.5 kg of oil had been recovered, a vivid pink-orange coloration of the effluent that left the separator was visually observed, thereby confirming our conclusion that not all the oil that was contained in the decanter effluent had been completely recovered.

A second test E was carried out on the same day and under the same conditions, except that the decanter effluent was heated by adding steam. The temperature of the effluent varied from 80 to 100° F. After the extraction system had been operating for 20 minutes, a subsample of each matrix (decanter effluent, sludge, separator effluent and oil) was taken in 15 minute intervals. This operation was repeated three times so as to obtain 3 subsamples of each matrix. The 3 subsamples of each matrix were mixed in order to obtain a composite sample so as to thus obtain more representative samples and to reduce the costs of the analyses. The composite samples of sludge and of shrimp oil were used to evaluate the quality of the final products obtained during all the experiments of this second step. The composite samples of decanter effluent (Dec EFF) and of separator effluent (Sep EFF) were used to evaluate the separator operating efficiency. The parameters that were determined on the composite samples of the decanter and separator effluents are the TS, TSS, CP and F contents. The results of these analyses are given in Table 5.

TABLE 5
Results of the evaluation of the separator
efficiency during the experiments of test E
Separator optimization (test E)
(Decanter 75%; effluent flow rate = 38 l/min;
effluent temperature: 80 to 100° F.)

| | Total | Total sus- | | Crude |
|---|---|---|---|---|
| | solids | pended solids | Fats | proteins |
| Sample | (TS) mg/l | (TSS) mg/l | (F) mg/l | (CP) mg/l |
| Dec Eff exp. E (IN) | 48 195 | 34 000 | 30 110 | 4925 |
| Sep Eff exp. E (OUT) | 31 109 | 14 800 | 14 525 | 4443 |
| Recovery (IN − OUT) | 17 086 | 19 200 | 15 585 | 482 |

Estimated recovery at the processing plant: 46 kg of oil/2635 liters of effluent or 17 457 mg of oil per liter of effluent
Density = 0.929 kg/l; (46 kg/49.5 liters)

According to the results of the analyses given in Table 5, it is noted that the decanter effluent is very rich in suspended solids, which consist mainly of fats and proteins.

When observing the results in Table 5, it can be noted that there is a small contradiction between the recovered amounts of TS (17 086 mg/l) and of TSS (19 200 mg/l). This contradiction shows us that, despite our efforts to prepare composite samples from 3 subsamples, the decanter and separator effluents remain very heterogeneous and difficult to analyze. Using the results of analyses obtained as a basis, it can be noted that a minimum of 15 585 mg of shrimp oil per liter of effluent were recovered at the time of the sampling. During this experiment, by centrifuging 2635 liters of effluent, 46 kg of oil were extracted, which represents an average recovery rate of 17 457 mg of oil per liter of effluent centrifuged. This experiment also demonstrated that increasing the decanter effluent temperature between 80 and 100° F. made it possible to increase the oil recovery rate. In order to further improve the oil extraction yield, it was therefore decided to carry out another experiment, but this time by heating the effluent to a higher temperature.

Consequently, a third experiment at the processing plant was carried out (F), this time using the decanter effluent heated to 175° F. while keeping the other parameters constant, such as the rotational speed of the decanter bowl at 2700 rpm and the pumping of the effluent into the separator with a flow rate of 38 liters per minute. The results of this experiment are given in Table 6.

TABLE 6
Results of the evaluation of the separator efficiency during experiment F
Test F, decanter speed = 2700 rpm; effluent flow rate = 38 l/min;
effluent temperature = 175° F.

| | Total | Total sus- | | Crude |
|---|---|---|---|---|
| | solids | pended solids | Fats | proteins |
| Sample | (TS) mg/l | (TSS) mg/l | (F) mg/l | (CP) mg/l |
| Dec Eff exp. F | 49 049 | 34 400 | 31 710 | 4844 |
| Sep Eff exp. F | 20 514 | 7125 | 6030 | 4338 |
| Recovery | 28 535 | 27 275 | 25 680 | 506 |

Estimated recovery at the processing plant: 43.5 kg of oil/2350 liters of effluent or 18 511 mg of oil per liter of effluent
The density is 0.929 kg/l; (43.5 kg/46.8 liters)

The results of the analyses demonstrated that increasing the decanter effluent temperature greatly improved the TS, TSS and F recovery efficiency. The results of the analyses are coherent and confirm that, during this test and at the time of the sampling (i.e. the extraction system had operated for 1 hour), a minimum of 25 680 mg of oil per liter of effluent were recovered. During this experiment (F), by centrifuging 2350 liters of effluent, 43.5 kg of oil were extracted, which represents an average recovery rate of 18 511 mg of oil per liter of effluent centrifuged. This demonstrates an extraction yield that is improved compared with that obtained during test E (17 457 mg of oil per liter of effluent), but which is lower than that which was estimated in the laboratory (25 680 mg of oil per liter of effluent). It is normal for the yield estimated in the laboratory to be higher than the yield actually obtained once the extraction process in the processing plant is finished, since the composite samples of effluents that were used to determine the extraction yield were taken at the time when the operation of the extraction system equipment was stabilized, which can probably lead to an overestimation of the yield. Furthermore, the operation of the separator is periodically disturbed during the discharge of the solids, which can also have a downward influence on the yield. Nevertheless, this test demonstrated that raising the effluent temperature up to 175° F. constitutes a not insignificant parameter which can improve the shrimp oil extraction yield. Consequently, the extraction system operating conditions used in this test for extracting the oil were considered to be satisfactory for application thereof on a larger scale.

The fourth test G was carried out while attempting to apply the same conditions as those used in test F. The objective of this test G was to evaluate the large-scale operation of the shrimp oil extraction system. During this experiment, it was not possible to heat the effluent to the planned temperature of 175° F., because of a large production volume. The effluent temperature only reaches 140° F. The extraction system operated in continuous mode and the decanter effluent was directly introduced into the separator with a variable flow rate between 38 and 46 liters per minute. The samples were taken for laboratory analyses and the total volume of effluent centrifuged and also the total amount of oil recovered were measured at the processing plant in order to evaluate the yield. The results of this test are given in Table 7.

TABLE 7
Results of the evaluation of the separator efficiency during experiment G
Test G, Decanter speed = 2700 rpm; effluent pump: from 38 to 46 l/min;
effluent temperature: 140° F.

| | Total | Total sus- | | Crude |
|---|---|---|---|---|
| | solids | pended solids | Fats | proteins |
| Sample | (TS) mg/l | (TSS) mg/l | (F) mg/l | (CP) mg/l |
| Dec Eff exp. Gt | 48 609 | 33 450 | 25 185 | 7806 |
| Sep Eff exp. G | 31 497 | 16 100 | 11 610 | 6281 |
| Recovery | 17 112 | 17 350 | 13 575 | 1525 |

Estimated recovery in the processing plant: 363 kg of oil/36 015 liters of effluent or 10 079 mg of oil per liter of effluent
The density is 0.931 kg/l; (363 kg/390 liters)

The results obtained during this large-scale experiment did not live up to our expectations. The extraction yield estimated in the laboratory was only 13 575 mg of oil per liter of effluent. The yield obtained in the processing plant was no better and confirmed this decrease compared to that obtained during test F. It was on average 10 079 mg of oil per liter of effluent centrifuged, whereas that obtained during test F was on average 18 511 mg of oil per liter. In order to explain this decrease in yield, the results of the analyses represented in Table 7 were examined more closely. They show that the decanter effluent contained pretty much the same amounts of solids as those that were treated in the previous tests. On the other hand, the amount of crude proteins was higher, while the amount of fats was lower. Furthermore, it was noted that, during this large-scale test (36 015 liters), the operation of the separator was disturbed because of the problem of discharging the solids.

The separator was cleaned and a final test H was carried out in order to reevaluate the oil extraction yield, but this time by treating a small amount of effluent (small-scale treatment) while trying to apply the same conditions as those used in test G. The decanter effluent was heated to the temperature of 140° F. and pumped into the separator with a variable flow rate of between 38 and 46 liters per minute. The results obtained during this test are given in Table 8.

TABLE 8
Results of the evaluation of the separator efficiency during experiment H
Test H; decanter = 2700 rpm; effluent pump: from 38 to 46 l/min;
effluent temperature: 140° F.

| | Total | Total sus- | | Crude |
|---|---|---|---|---|
| | solids | pended solids | Fats | proteins |
| Sample | (TS) mg/l | (TSS) mg/l | (F) mg/l | (CP) mg/l |
| Dec Eff exp. H | 55 779 | 40 600 | 32 590 | 8919 |
| Sep Eff exp. H | 27 834 | 14 250 | 10 490 | 4913 |
| Reduction/recovery | 27 945 | 26 350 | 22 100 | 4006 |

Estimated recovery in the processing plant: 48 kg of oil/2344 liters of effluent or 20 478 mg of oil per liter of effluent
The density is 0.923 kg/l; (48 kg = 52 liters)

The results of the laboratory analyses demonstrated that, during this test, it was possible to extract 22 100 mg of oil per liter of effluent. The evaluation of the yield in the processing plant confirmed that the small-scale extraction system operating efficiency was good. 20 478 mg of oil were on average extracted per liter of effluent centrifuged, even though the decanter effluent contained larger amounts of solids, of crude proteins and of fats than in the previous tests. This result shows that the extraction system equipment is suitable for the treatment of a medium volume of effluent, i.e. when the extraction system operates over a short period of time. In order to treat large effluent volumes, i.e. when the extraction system operates in continuous mode over a long period of time, it would probably be necessary to decrease the rotational speed of the decanter bowl in order to have an effluent with a lower solids content, which may facilitate the discharge of the solids accumulated in the separator and thus improve the oil extraction efficiency.

The results obtained during these tests made it possible to determine the parameters for obtaining good separator operation and thus obtaining a good oil extraction yield:
1. The rotational speed of the decanter bowl can be decreased to 2520 rpm (a decanter operating rate equal to 70% of its maximum speed) and even down to 2160 rpm (60% of the maximum speed) in order to have an effluent with a lower solids content and thus to avoid their accumulation in the separator.
2. The effluent should be pumped into the separator with a flow rate of 38 to 46 liters per minute or 2280 to 2760 l/hour (a pump operating rate between 45% and 55% of its maximum flow rate).
3. The decanter effluent should be preheated to a minimum temperature of 140° F. using steam (ideally the decanter effluent should be preheated to a temperature of 95° C. using the heat exchanger).
4. In order to produce this oil on a large scale it would be necessary to acquire 1) a separator more suitable for this process and 2) the heat exchanger for preheating the decanter effluent to a temperature of 95° C.

### Example 4

### Characterization of the Products Obtained

### Characterization of the Oil Recovered

The total fatty acid profile was determined on each sample of shrimp oil extracted during experiments E, F, G and H in order to evaluate its quality. The results of this profile are given in Table 9.

TABLE 9
Total fatty acid profile of the shrimp oil extracted during the experiments

| | | | | | | | Standard |
|---|---|---|---|---|---|---|---|
| | test E | test F | test G | test H | Mean | | deviation |
| Fatty acid | g/100 g | g/100 g | g/100 g | g/100 g | g/100 g | % | g/100 g |
| C14:0 | 3.73 | 3.75 | 3.91 | 3.99 | 3.84 | 4.6 | 0.13 |
| C15:0 | 0.33 | 0.34 | 0.30 | 0.30 | 0.32 | 0.38 | 0.02 |
| C16:0 | 11.94 | 12.35 | 12.13 | 12.11 | 12.13 | 15 | 0.17 |
| C18:0 | 2.46 | 2.51 | 2.53 | 2.50 | 2.50 | 3.0 | 0.03 |
| C20:0 | 0.12 | 0.11 | 0.11 | 0.11 | 0.11 | 0.13 | 0.00 |
| C22:0 | 0.00 | 0.00 | 0.00 | 0.00 | 0.00 | 0 | 0.00 |
| C24:0 | 0.00 | 0.00 | 0.00 | 0.00 | 0.00 | 0 | 0.00 |
| C14:1 | 0.33 | 0.34 | 0.33 | 0.35 | 0.34 | 0.41 | 0.01 |
| C16:1 | 11.61 | 11.94 | 11.54 | 11.15 | 11.56 | 14 | 0.32 |
| C18:1 | 14.97 | 15.58 | 15.29 | 15.39 | 15.31 | 18.3 | 0.26 |
| C20:1 | 7.47 | 7.19 | 6.59 | 7.37 | 7.15 | 8.6 | 0.39 |
| C22:1 | 8.84 | 7.39 | 8.24 | 9.32 | 8.44 | 10 | 0.83 |
| C24:1 | 0.30 | 0.28 | 0.28 | 0.27 | 0.28 | 0.34 | 0.02 |
| C18:2n6 | 0.73 | 0.81 | 0.81 | 0.87 | 0.81 | 0.97 | 0.06 |
| C20:2n6 | 0.30 | 0.30 | 0.22 | 0.24 | 0.26 | 0.31 | 0.04 |
| C22:2n6 | 0.00 | 0.00 | 0.00 | 0.00 | 0.00 | 0 | 0.00 |
| C18:3n6 | 0.00 | 0.00 | 0.00 | 0.00 | 0.00 | 0 | 0.00 |
| C18:3n3 | 0.59 | 0.60 | 0.67 | 0.79 | 0.66 | 0.79 | 0.09 |
| C20:3n6 | 0.15 | 0.13 | 0.13 | 0.14 | 0.14 | 0.17 | 0.01 |
| C20:3n3 | 0.12 | 0.12 | 0.11 | 0.12 | 0.12 | 0.14 | 0.01 |
| C18:4n3 | 1.27 | 1.38 | 1.45 | 1.56 | 1.41 | 1.7 | 0.12 |
| C20:4n6 | 0.42 | 0.47 | 0.36 | 0.35 | 0.40 | 0.48 | 0.06 |
| C20:4n3 | 0.37 | 0.33 | 0.31 | 0.30 | 0.33 | 0.40 | 0.03 |
| C22:4n6 | 0.19 | 0.20 | 0.19 | 0.17 | 0.19 | 0.23 | 0.02 |
| C20:5n3 (EPA) | 8.87 | 8.77 | 8.49 | 8.45 | 8.64 | 10 | 0.21 |
| C22:5n6 | 0.15 | 0.16 | 0.05 | 0.11 | 0.12 | 0.14 | 0.05 |
| C22:5n3 | 0.46 | 0.46 | 0.35 | 0.35 | 0.40 | 0.48 | 0.06 |
| C22:6n3 (DHA) | 7.83 | 7.56 | 8.28 | 8.55 | 8.05 | 9.6 | 0.44 |
| Total | 83.55 | 83.07 | 82.68 | 84.83 | 83.53 | 100 | 0.94 |
| Saturated | 18.57 | 19.06 | 18.99 | 19.01 | 18.91 | 23 | 0.23 |
| Monounsaturated | 43.52 | 42.71 | 42.27 | 43.85 | 43.09 | 52 | 0.73 |
| Polyunsaturated | 21.45 | 21.29 | 21.43 | 21.97 | 21.54 | 26 | 0.30 |
| Total fat | 87.27 | 86.77 | 86.36 | 88.60 | 87.25 | | 0.97 |
| Omega-3 | 19.51 | 19.22 | 19.66 | 20.10 | 19.62 | 23 | 0.37 |
| Omega-6 | 1.94 | 2.08 | 1.77 | 1.87 | 1.91 | 2.3 | 0.13 |

The results in Table 9 demonstrate that the profile of fatty acids contained in the oil is similar in all the samples extracted during the experiments. The oil recovered is of good quality since it contains a large amount of omega-3 acids (23%), including 10% of EPA (Eicosapentaenoic Acid Methyl Ester) and 9.6% of DHA (Docosahexaenoic Acid Methyl Ester). Furthermore, the analyses carried out on these samples demonstrate that the oil obtained contains no trace of moisture. This oil can therefore constitute a food additive that has very advantageous characteristics, inter alia for incorporation thereof into aquaculture feeds and especially those intended for salmonids.

### Physicochemical Characterization of the Organic Sludge Recovered

The samples of the organic sludge recovered after use of the decanter during the experiments were analyzed in order to determine their moisture content, total solids content, fat content, crude protein content and ash content. The results of these analyses are given in Table 10.

TABLE 10
Results of the analyses of the composite samples of
sludge recovered during experiments E, F, G and H

| | Moisture | Total | Crude | | |
|---|---|---|---|---|---|
| | content | solids | proteins | Fat | Ash |
| Sample | % | % | % | % | % |
| E | 81.23 | 18.77 | 11.44 | 2.39 | 3.88 |
| F | 82.98 | 17.02 | 11.75 | 2.26 | 2.51 |
| G | 82.17 | 17.83 | 11.38 | 1.39 | 3.07 |
| H | 82.19 | 17.81 | 10.63 | 1.89 | 3.27 |
| Mean | 82.14 | 17.86 | 11.30 | 1.98 | 3.27 |
| Standard | 0.72 | 0.72 | 0.48 | 0.45 | 0.57 |
| deviation | | | | | |

According to the results of the analyses of Table 10, it is seen that the chemical composition of the sludge is constant for all the samples taken during the experiments. They consist on average of 82.14±0.72% moisture content and 17.86±0.72% total solids. The total solids consist on average of 1.98±0.45% fats, 11.30±0.48% crude proteins and 3.27±0.53% ash. These results indicate that the extraction of the oil did not affect the quality of the sludge recovered during these tests since its chemical composition is comparable to that obtained during the previous tests.

In order to evaluate the quality of the fats contained in the sludge recovered, the total fatty acid profile was determined. The results of this profile are given in Table 11.

TABLE 11
Total profile of the fatty acids contained in the sludge recovered
from the decanter during experiments E, F, G and H

| | | | | | | | Standard |
|---|---|---|---|---|---|---|---|
| | E | F | G | H | Mean | | deviation |
| Fatty acid | g/100 g | g/100 g | g/100 g | g/100 g | g/100 g | % | g/100 g |
| C14:0 | 0.10 | 0.10 | 0.10 | 0.09 | 0.10 | 3.6 | 0.01 |
| C15:0 | 0.01 | 0.01 | 0.01 | 0.01 | 0.01 | 0.36 | 0.00 |
| C16:0 | 0.45 | 0.49 | 0.43 | 0.41 | 0.45 | 16 | 0.04 |
| C18:0 | 0.09 | 0.10 | 0.08 | 0.08 | 0.09 | 3.2 | 0.01 |
| C20:0 | 0.00 | 0.01 | 0.00 | 0.00 | 0.00 | 0 | 0.00 |
| C22:0 | 0.01 | 0.01 | 0.01 | 0.01 | 0.01 | 0.36 | 0.00 |
| C24:0 | 0.00 | 0.00 | 0.00 | 0.00 | 0.00 | 0 | 0.00 |
| C14:1 | 0.01 | 0.01 | 0.01 | 0.01 | 0.01 | 0.36 | 0.00 |
| C16:1 | 0.32 | 0.35 | 0.31 | 0.29 | 0.32 | 12 | 0.02 |
| C18:1 | 0.52 | 0.61 | 0.52 | 0.50 | 0.54 | 20 | 0.05 |
| C20:1 | 0.15 | 0.21 | 0.14 | 0.13 | 0.16 | 5.8 | 0.03 |
| C22:1 | 0.15 | 0.19 | 0.14 | 0.13 | 0.15 | 5.5 | 0.02 |
| C24:1 | 0.02 | 0.02 | 0.01 | 0.01 | 0.02 | 0.73 | 0.00 |
| C18:2n6 | 0.03 | 0.04 | 0.03 | 0.03 | 0.03 | 1.1 | 0.00 |
| C20:2n6 | 0.01 | 0.01 | 0.01 | 0.01 | 0.01 | 0.36 | 0.00 |
| C22:2n6 | 0.00 | 0.00 | 0.00 | 0.00 | 0.00 | 0 | 0.00 |
| C18:3n6 | 0.00 | 0.00 | 0.00 | 0.00 | 0.00 | 0 | 0.00 |
| C18:3n3 | 0.02 | 0.03 | 0.02 | 0.02 | 0.02 | 0.73 | 0.00 |
| C20:3n6 | 0.00 | 0.00 | 0.00 | 0.00 | 0.00 | 0 | 0.00 |
| C20:3n3 | 0.00 | 0.01 | 0.00 | 0.00 | 0.00 | 0 | 0.00 |
| C18:4n3 | 0.04 | 0.04 | 0.04 | 0.04 | 0.04 | 1.5 | 0.00 |
| C20:4n6 | 0.03 | 0.04 | 0.03 | 0.03 | 0.03 | 1.1 | 0.00 |
| C20:4n3 | 0.01 | 0.01 | 0.01 | 0.01 | 0.01 | 0.36 | 0.00 |
| C22:4n6 | 0.00 | 0.01 | 0.00 | 0.01 | 0.00 | 0 | 0.00 |
| C20:5n3 (EPA) | 0.38 | 0.42 | 0.40 | 0.38 | 0.39 | 14 | 0.02 |
| C22:5n6 | 0.00 | 0.00 | 0.00 | 0.00 | 0.00 | 0 | 0.00 |
| C22:5n3 | 0.01 | 0.02 | 0.01 | 0.01 | 0.01 | 0.36 | 0.00 |
| C22:6n3 (DHA) | 0.33 | 0.35 | 0.33 | 0.31 | 0.33 | 12 | 0.01 |
| Total | 2.71 | 3.06 | 2.66 | 2.51 | 2.74 | 100 | 0.23 |
| Saturated | 0.66 | 0.72 | 0.63 | 0.59 | 0.65 | 24 | 0.05 |
| Monounsaturated | 1.17 | 1.38 | 1.13 | 1.07 | 1.19 | 43 | 0.13 |
| Polyunsaturated | 0.88 | 0.97 | 0.89 | 0.85 | 0.90 | 33 | 0.05 |
| Total fat | 2.83 | 3.20 | 2.77 | 2.63 | 2.86 | 100 | 0.24 |
| Omega-3 | 0.81 | 0.87 | 0.81 | 0.77 | 0.82 | 30 | 0.04 |
| Omega-6 | 0.07 | 0.10 | 0.08 | 0.08 | 0.08 | 2.9 | 0.01 |

The results given in Table 11 demonstrate that the total profile of the fatty acids contained in the sludge is similar for all the samples recovered during the experiments E, F, G and H. The fat contained in the sludge is of very good quality. It contains a large amount of omega-3 acids (30%), in particular 14% of EPA (Eicosapentaenoic Acid Methyl Ester) and 12% of DHA (Docosahexaenoic Acid Methyl Ester) which play an important role in nutrition. It is important to emphasize that the sludge recovered has a slight dull pink tint which attests to the presence of a carotenoid pigment, which adds value to it with regard to its incorporation into animal nutrition and especially into aquaculture feeds.

### Example 5

### Summary of the Conditions

In order to obtain a greater amount of oil and a smaller amount of proteins in the shrimp effluent without harming the quality of the organic sludge recovered, the optimal conditions for the two-phase horizontal centrifuge (decanter) were established. The rotational speed of the decanter bowl is a parameter which has a great influence on the separation of the solids from the liquid. Experiments were carried out in order to evaluate the decanter operating efficiency at 7 different bowl rotational speeds. The variations in the quality of the shrimp processing effluent and in the DAF operating efficiency were also evaluated. The results of these experiments demonstrated that:
The quality of the shrimp effluent varied slightly during these experiments. The total suspended solids (TSS) content varied between 1404 and 2191 mg/l, while the total nitrogen content (400 to 462 mg/l), crude protein content (2500 to 2890 mg/l) and fat content (1013 to 1099 mg/l) varied little.
The percentage reductions in TSS (78% to 85%), TKN (28% to 32%), CP (28% to 32%) and F (60% to 69%) in the shrimp processing effluent demonstrate that the DAF system operates with the same efficiency that was obtained during the months of September and October 2005 after its optimization was finalized.
Changing the rotational speed of the decanter bowl does not significantly affect the quality of the organic sludge recovered.
Increasing the rotational speed of the decanter bowl promotes an increase in solids (TS and TSS), crude proteins and fat in the effluent.
The decanter effluent samples that were taken during the operation of the decanter at bowl rotational speeds greater than 2700 rpm (75% of the maximum speed) are not suitable for the production of shrimp oil since they contain too great an amount of solids and of proteins.
The samples that were treated by operating the decanter at a rotational speed of 1800 rpm (the slowest speed tested in this project) have a lower fat concentration despite the fact that their solids and protein contents are perfect for operating the separator.
The decanter effluent samples that were taken during the operation of the decanter at bowl rotational speeds of 2160, 2520 and 2700 rpm (i.e. 60%, 70% and 75% of the maximum speed, respectively) appear to be the most suitable for the extraction of shrimp oil since they contained a good amount of fats and moderate amounts of solids and especially of proteins.

The optimization of the process for extraction of the oil that is in the decanter effluent, using the separator, made it possible to establish that:
The decanter effluent should be preheated to a minimum temperature of 140° F. using steam (ideally the decanter effluent should be preheated to a temperature of 95° C. using the heat exchanger).
The effluent should be pumped into the separator with a flow rate of 38 to 46 liters per minute or 2280 to 2760 l/hour (a pump operating rate between 45% and 55% of its maximum flow rate).
The rotational speed of the decanter bowl can be decreased to 2520 rpm (a decanter operating rate equal to 70% of its maximum speed) and even down to 2160 rpm (60% of the maximum speed) in order to have an effluent with a lower solids content and thus to avoid their accumulation in the separator.
By adhering to the parameters indicated above, it is possible to extract on average 20 478 mg of oil per liter of effluent.
To produce oil on a large scale it is necessary to acquire 1) a separator that is more suitable for this process and 2) a heat exchanger that makes it possible to preheat the decanter effluent to a temperature of 95° C.

The optimization of the operation of the decanter and of the separator made it possible to recover organic sludge and shrimp oil. The results of the chemical analyses of the organic sludge and of the oil recovered from the shrimp effluents made it possible to note that:
The sludge recovered after use of the decanter contains on average 17.86±0.72% of total solids which consist of 1.98±0.45% of fat, 11.30±0.48% of crude proteins and 3.27±0.53% of ash. The fat contained in the sludge is of very good quality. It contains a large amount of omega-3 acids (30%), in particular 14% of EPA (Eicosapentaenoic Acid Methyl Ester) and 12% of DHA (Docosahexaenoic Acid Methyl Ester), which plays an important role in nutrition. It is important to emphasize that the sludge recovered has a slight dull pink tint which attests to the presence of a carotenoid pigment, which adds value to it with regard to its incorporation into aquaculture feeds. The results of the analyses of the lyophilized organic sludge, referred to as shrimp organic solids (SOC), are given in Table 12.
The shrimp oil obtained is very rich in omega-3 fatty acid and in astaxanthin (Table 12), a carotenoid pigment which is responsible for the pink-orange coloration of the flesh of salmonids and crustaceans (crab, shrimp, lobster). Furthermore, the total fatty acid profile shows that the shrimp oil extracted is of very good quality because it contains a large amount of omega-3 acids (23%), including 10% of EPA (Eicosapentaenoic Acid Methyl Ester) and 9.6% of DHA (Docosahexaenoic Acid Methyl Ester). Furthermore, the oil obtained contains no trace of moisture. This oil can therefore constitute a food additive which has very advantageous characteristics, inter alia for the incorporation thereof into aquaculture feeds and especially those intended for salmonids.

TABLE 12
Characteristics of the shrimp organic solids and of the shrimp oil

| | | Shrimp organic solids | Shrimp |
|---|---|---|---|
| Analysis | | (lyophilized sample) | oil |
| Crude proteins | % | 65 | — |
| Fats | % | 13.5 | |
| Moisture content | % | 80 | — |
| (wet sample) | | | |
| Moisture content | % | 0 | — |
| Ash | % | 14 | — |
| Vitamin E | μg/g | 425 | 575 |
| Vitamin A | IU/100 g | 4700 | 2200 |
| Cholesterol | mg/100 g | 367 | 26.3 |
| Phospholipids | g/100 g | 4 | — |
| Total astaxanthin | μg/g | 358 | 859 |
| Di-cis-astaxanthin | μg/g | 18 | 37 |
| All-trans astaxanthin | μg/g | 295 | 567 |
| (3S,3′S)-9-cis-astaxanthin | μg/g | 12 | 71 |
| (3S,3′S)-13-cis-astaxanthin | μg/g | 33 | 184 |

### Example 6

### Study of the Effect of Temperature on the Separation of the Shrimp Organic Solids (SOC) and of the Shrimp Oil Using the Decanter

The shrimp processing process comprises several steps (cooking, cooling, shelling, inspection, pickling in brine, draining, freezing, etc.) and requires a very large amount of drinking water. The water used during the shrimp production (EPC) contains fats and proteins. The EPC is used as a raw material in our novel process for extracting shrimp oil (HC) and shrimp organic solids (SOC). The water which leaves the shrimp processing process (EPC) is first collected in a reservoir (R1) and is then pumped into a dissolved air flotation (DAF) system. The EPC temperature can vary between 10° C. and 17° C. In the DAF system, the solids, composed mainly of proteins, fats and minerals (ash), are collected by means of a procedure referred to as “skimming”. The solids recovered, referred to as “skimmings” (SKIM), are collected in a second reservoir (R2 SKIM) and pumped into the decanter which separates them into two phases: a solid phase, referred to as shrimp organic solids (SOC), and a liquid phase, referred to as decanter effluent (DEC EFF). The efficiency of the separation and the chemical composition of the SOC and DEC EFF depend on several variables, but the SKIM temperature is the main parameter which affects the mobility of the oil. The objective of this study was therefore to determine the SKIM temperature and the rotational speed of the decanter bowl which make it possible to obtain the most oil-rich DEC EFF.

In a first test, 1810 1 of SKIM were accumulated in the reservoir R2. The SKIM was heated to a temperature of 85° C. with addition of steam. The SKIMs (85° C.) were then pumped into the decanter at a speed of 50 l/min. The decanter was operated at 3 different speeds: 65% (2520 rpm), 75% (2700 rpm) and 100% (3240 rpm) of its maximum speed. A second test was carried out under the same conditions, but at a SKIM temperature of 10° C. (natural temperature of production water). During these experiments, the SKIM, SOC and DEC EFF samples were taken in order to analyze the total solids (TS), ash, crude protein (CP) and fat (F) concentrations thereof. The results of these analyses are given in Table 13.

TABLE 13
Analyses of the samples taken during the tests at 85° C. and 10° C.

| | 85° C. | | | | 10° C. | | | |
|---|---|---|---|---|---|---|---|---|
| | TS | ash | CP | F | TS | ash | CP | F |
| | % | | | | % | | | |
| SKIM a | 6.36 | 1.57 | 2.80 | 1.89 | 5.41 | 1.38 | 3.05 | 1.22 |
| SKIM b | 6.25 | 1.69 | 2.73 | 1.77 | 5.79 | 72 | 2.68 | 1.55 |
| SKIM c | 5.82 | 1.63 | 2.45 | 1.68 | 5.57 | 1.66 | 2.55 | 1.51 |
| SOC | 30.7 | 4.40 | 13.8 | 12.2 | 17.0 | 3.40 | 9.28 | 4.13 |
| a (65% Max | | | | | | | | |
| Speed) | | | | | | | | |
| SOC | 35.6 | 5.00 | 15.7 | 15.0 | 16.7 | 3.40 | 9.09 | 3.88 |
| b (75% Max | | | | | | | | |
| Speed) | | | | | | | | |
| SOC c (Max | 35.0 | 4.80 | 15.4 | 15.8 | 16.1 | 3.40 | 9.63 | 3.00 |
| Speed) | | | | | | | | |
| | mg/l | | | | Mg/l | | | |
| DEC EFF | 15 650 | 9190 | 1319 | 141 | 20 150 | 10 950 | 2579 | 5085 |
| a (65% Max | | | | | | | | |
| Speed) | | | | | | | | |
| DEC EFF | 15 500 | 9 040 | 1313 | 96 | 27 700 | 11 200 | 3694 | 11 400 |
| b (75% Max | | | | | | | | |
| Speed) | | | | | | | | |
| DEC EFF | 15 600 | 9 360 | 1063 | 126 | 31 800 | 11 200 | 5106 | 14 900 |
| c (Max Speed) | | | | | | | | |

### Composition of the Decanter Effluents (DEC EFF)

The results obtained during the tests at 85° C. and 10° C. made it possible to note that the SKIM temperature greatly affects the DEC EFF and SOC composition. At 10° C., DEC EFF recovered contains more solids than at 85° C. The ash values are affected very little, while the protein concentrations increase from 1319, 1313 and 1063 mg/l (10° C.) to 2579, 3694 and 5106 mg/l (85° C.) for decanter speeds of 65%, 75% and 100%, respectively.

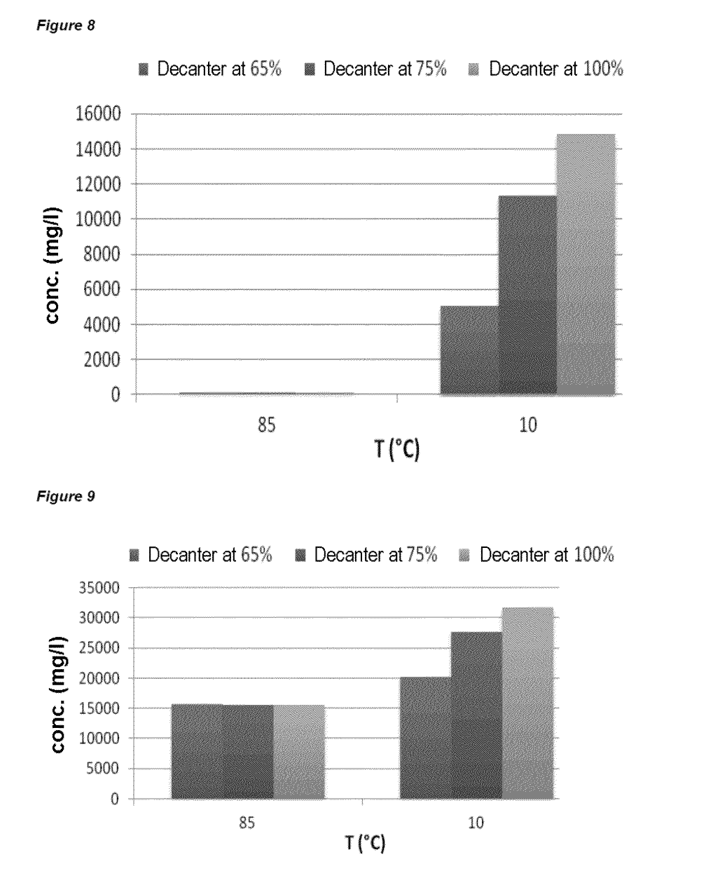

FIG. 8 clearly demonstrates that the temperature has a considerable effect on the concentration of fat (oil) contained in the decanter effluents. At 85° C., the fat concentrations of the effluents recovered were 148, 96 and 126 mg/l, while at 10° C., the concentrations were 6070, 11 370 and 14 880 mg/l, for decanter speeds of 65%, 75% and 100%, respectively.

FIG. 9 demonstrates the effect of the temperature and of the decanter speed on the concentration of total solids recovered. At 85° C., the decanter speed appears to have little effect on the concentration of total solids, which is 15 610, 15 510 and 15 580 mg/l for decanter speeds of 65%, 75% and 100%, respectively. The results obtained at a temperature of 10° C. demonstrate the effect of the decanter speed, where concentrations of 21 340, 27 720 and 31 790 mg/l are obtained for decanter speeds of 65%, 75% and 100%.

The results clearly demonstrate that a lower SKIM temperature makes it possible to obtain a decanter effluent that is richer in organic material and particularly richer in oil.

### Composition of the Shrimp Organic Solids Recovered (SOC)

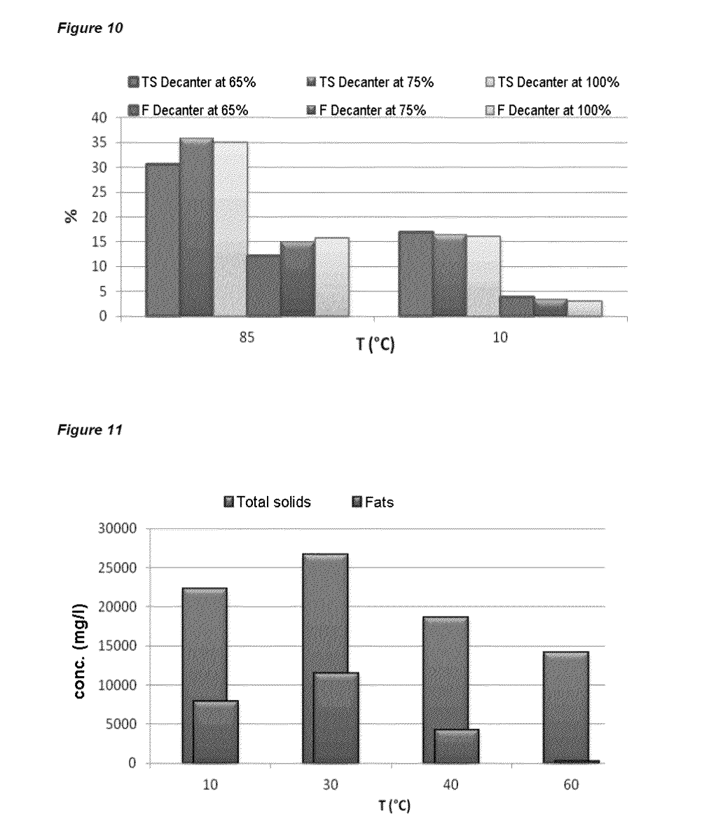

The total solids contained in the SKIM coming from the DAF system are distributed in the liquid and solid phases. FIG. 10 demonstrates the effect of the temperature and also of the decanter speed on the SOC composition. It is interesting to note that the trends are opposite to those observed for the effluents. At a temperature of 85° C., the SOC are richer in fats, with values of 12.4%, 15.0% and 15.7% for decanter speeds of 65%, 75% and 100%, respectively. At a temperature of 10° C., concentrations of 3.6%, 3.4% and 3.0% are obtained for the same decanter speeds. The same trend is observed for the total solids and the proteins.

The temperature has an enormous influence on the fat concentration of the effluents and of the solids. However, as only two temperatures were selected for these tests, a second experiment was carried out at intermediate temperatures.

This second experiment follows on from the results obtained during the first experiment in order to determine the temperature of the SKIMs which make it possible to obtain the most oil-rich decanter effluent (DEC EFF). Intermediate temperatures of 10° C., 30° C., 40° C. and 60° C. were selected and the decanter operates at 75% of its maximum speed (2700 rpm). These tests were carried out under the same conditions as the previous tests. The SOC and DEC EFF samples were taken in order to analyze the total solids (TS), crude protein (CP) and fat (F) concentrations thereof. The results of these analyses are given in Table 14.

TABLE 14
Analyses of the DEC EFF and SOC samples for
various temperatures; decanter at 2700 rpm

| | TS | CP | F |
|---|---|---|---|
| | % | | |
| SOC 10° C. | 17.5 | 9.62 | 4.80 |
| SOC 30° C. | 20.0 | 10.4 | 4.18 |
| SOC 40° C. | 22.4 | 11.0 | 5.83 |
| SOC 60° C. | 24.0 | 11.6 | 9.09 |
| | mg/l | | |
| DEC EFF 10° C. | 22 300 | 3031 | 7 870 |
| DEC EFF 30° C. | 26 600 | 5631 | 11 500 |
| DEC EFF 40° C. | 18 600 | 4463 | 4 210 |
| DEC EFF 60° C. | 14 300 | 4150 | 236 |

### Effect of the Temperature on the Composition of the Decanter Effluent (DEC EFF)

FIG. 11 demonstrates the effect of the temperature on the fat and total solids concentration of the decanter effluents (DEC EFF). It is interesting to note that the increase in the fat concentration goes from 7870 mg/l to 11 500 mg/l when the temperature is raised from 10° C. to 30° C. This represents a 42% increase. It can also be seen that the fat concentration reaches a maximum value at 30° C. and then decreases and reaches a minimum value at 60° C. A similar, although less marked, trend is observed for the total solids concentration as a function of the temperature. This demonstrates that the migration of the fat to the liquid phase (the effluent) is promoted at a temperature of approximately 30° C.

### Effect of the Temperature on the Composition of the Shrimp Organic Solids (SOC)

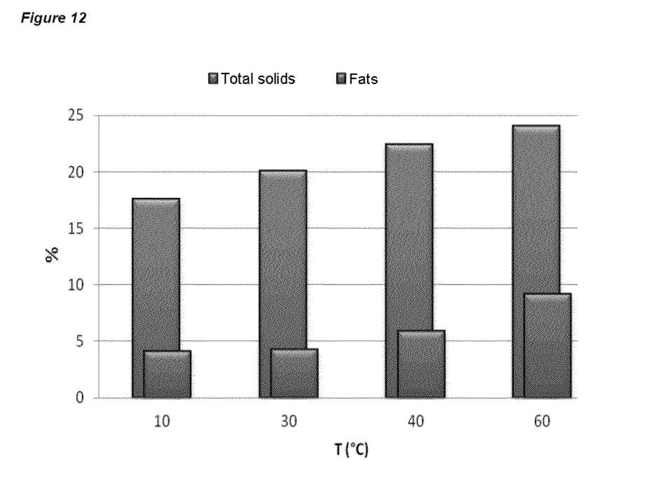

In FIG. 12, the effect of the temperature on the composition of fats and the total solids of the SOC is demonstrated. In both cases, an upward, almost linear trend is obtained, contrary to what is observed for the decanter effluent. It therefore appears that a higher temperature promotes the recovery of organic solids (SOC) rather than the recovery of the oil.

### Conclusion

This study demonstrated that: a) increasing the “SKIM” temperature to approximately 30° C. makes it possible to obtain a decanter effluent that is richer in oil; and b) increasing the “SKIM” temperature to approximately 85° C. makes it possible to obtain the shrimp organic solids richer in fats and in proteins.

### Example 7

Development of a process for producing shrimp oil and shrimp organic solids (SOC) based on the water used in shrimp processing

Multiple studies on a laboratory scale and on a pilot processing plant scale made it possible to develop the process for producing novel products based on water used in shrimp processing in order to obtain an astaxanthin-rich shrimp oil and shrimp organic solids (SOC).

FIG. 1 demonstrates the diagram of the process for producing shrimp oil and shrimp organic solids (SOC) based on water used in shrimp processing. Our novel industrial-scale process comprises several steps and requires the following pieces of equipment:

Reservoir #1: This reservoir is used to accumulate all the shrimp processing process effluents. The flow rate of the effluent leaving this reservoir is approximately 2000 l/min. The effluent contains approximately 1.8% of solids. It should be made of stainless steel and isolated from the external environment.

Dissolved air flotation (DAF) tank: The DAF system is a process which uses small air bubbles that attach to solid particles. These particles rise to the surface where they are collected by a skimming device. The solids recovered in this step are referred to as “skimmings” and contain approximately 6% of organic solids and 94% of water. The flow rate of the “skimmings” is 60-90 l/m.

Reservoir #2: Reservoir #2 is used to accumulate the “skimmings” recovered from the flotation tank DAFs. Reservoir #2 should be made of stainless steel and equipped with a mixer, in order to keep the solution homogeneous.

Pump #1: This is a pump which is used to transfer the “skimmings” from reservoir #2 to the heat exchanger #1. The flow rate of the “skimmings” is approximately 60-90 l/min.

Heat exchanger #1 (EC1): The heat exchanger is used to heat the “skimmings” before they go into the centrifugal decanter. This heat exchanger must be capable of rapidly increasing the temperature of the “skimmings” a) from 10° C. to 85° C. in order to improve the SOC production (quality, yield and profitability), and b) from 10° C. to 30° C. in order to improve the shrimp oil production.

Centrifuge decanter (DEC): The decanter is used to separate the solid phase (SOC) and the liquid phase (decanter effluent) which contains the oil.

Reservoir #3: Reservoir #3 is used to accumulate the decanter effluent. It should be made of stainless steel and equipped with a mixer, in order to keep the solution homogeneous.

Pump #2: Pump #2 is the same style of pump as pump #1. It is used to transfer the effluents from reservoir #3 to the second heat exchanger. Its flow rate will be approximately 50-60 l/min.

Heat exchanger #2 (EC2): The shrimp oil extraction process requires two heat exchangers since there are two different operations which require a heat exchanger at the same time. This piece of equipment will be used to heat the decanter effluent to 90±5° C.

Separator (SEP): The separator is a vertical centrifuge. This device is capable of separating the decanter effluent into three phases: the shrimp oil, the SOC and the separator effluent which contains very few solids.

1. A process for extracting oil of marine origin, comprising:
a) obtaining a marine organism treatment effluent;
b) adding a flocculating agent to the treatment liquid of a) and separating an aqueous phase from the solids flocculated at the surface in order to recover said solids therefrom;
c) separating said solids recovered in b) into a solid phase and a liquid phase and recovering the solid phase and/or the liquid phase;
d) subjecting the liquid phase obtained in c) to a vertical centrifugation in order to obtain an aqueous phase and an oil; and
e) recovering the oil thus separated.

2. The process as claimed in claim 1, wherein:
b) the separation of said liquid phase from said solids flocculated at the surface is carried out with a dissolved air flotation (DAF) system;
c) the separation of said solids flocculated in b) into a solid phase and a liquid phase is carried out using a 2-phase horizontal centrifuge; and
d) the obtaining of the aqueous phase and of the oil is carried out by subjecting the liquid phase obtained in c) to a vertical centrifugation.

3. The process as claimed in claim 1, also comprising the following step:
f) the solid phase recovered in c) is then dried in order to form a protein-enriched solid residue.

4. The process as claimed in claim 1, wherein the treatment liquid obtained in step a) is at a temperature of approximately 4 to 25° C. before carrying out step b).

5. The process as claimed in claim 1, wherein the solid phase obtained in step b) is heated to approximately 20 to 40° C. before carrying out step c).

6. The process as claimed in claim 1, wherein the liquid phase obtained in step c) is heated to a temperature of approximately 80 to 99° C. before carrying out step d).

7. The process as claimed in claim 2, wherein the decanting in step c) is carried out at a bowl rotational speed of between 1800 and 3300 rpm.

8. The process as claimed in claim 1, wherein the marine organism is chosen from: crustaceans and fish.

9. (canceled)

10. The process as claimed in claim 8, wherein the crustacean is shrimp.

11. The process as claimed in claim 1, wherein the treatment liquid is chosen from: cooking water, cooling water, rinsing water, washing water, shelling water, water from pickling in brine, and draining water.

12. An oil as obtained by the process as claimed in claim 1.

13. (canceled)

14. (canceled)

15. (canceled)

16. A marine organism solid residue comprising: more than 60% proteins, more than 400 μg/g of vitamin E, more than 4000 IU/100 g of vitamin A, and more than 350 μg/g of astaxanthin.

17. (canceled)

18. (canceled)

19. (canceled)

20. (canceled)

21. (canceled)

22. (canceled)

23. (canceled)

24. A composition comprising an oil as defined in claim 12, mixed with an excipient.

25. (canceled)

26. (canceled)

27. A feed intended for feeding at least one farm-raised fish, said feed comprising an oil as defined in claim 12.

28. A feed intended for feeding at least one farm-raised fish, said feed comprising a solid residue as defined in claim 16.

29. (canceled)

30. A food supplement comprising an oil as defined in claim 12, mixed with an excipient which is physiologically acceptable in animals.

31. A food supplement comprising a solid residue as defined in claim 16, mixed with an excipient which is physiologically acceptable in animals.

32. The food supplement as claimed in claim 30, wherein
the animal is a human being.

33. The food supplement as claimed in claim 30, wherein the animal is a pet.

34. The food supplement as claimed in claim 30, wherein the animal is a farm animal.

35. (canceled)

36. (canceled)

37. (canceled)

38. (canceled)

39. (canceled)

40. (canceled)
